SEQUENCE LISTING (31 sequences)

SEQ ID NO: 1 - RNA, 998 nt, synthetic construct
  source 1..998: mol_type = other RNA
gagggaaataagagagaaaagaagagtaagaagaaatataagagccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaagcggccgcttaattaagctgccttctgcggggcttgccttctggccatgcccttcttctctcccttgcacctgtacctcttggtctttgaataaagcctgagtaggaagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - RNA, 30 nt, synthetic construct
  source 1..30: mol_type = other RNA
gagggaaataagagagaaaagaagagtaag

SEQ ID NO: 3 - RNA, 30 nt, synthetic construct
  source 1..30: mol_type = other RNA
aatataagagccaccatggtgagcaagggc

SEQ ID NO: 4 - RNA, 30 nt, synthetic construct
  source 1..30: mol_type = other RNA
ttggtctttgaataaagcctgagtaggaag

SEQ ID NO: 5 - RNA, 120 nt, synthetic construct
  source 1..120: mol_type = other RNA
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - DNA, 30 bp, synthetic construct
  source 1..30: mol_type = other DNA
cttactcttcttttctctcttatttccctc

SEQ ID NO: 7 - DNA, 30 bp, synthetic construct; identical to SEQ ID NO: 6
  source 1..30: mol_type = other DNA

SEQ ID NO: 8 - DNA, 30 bp, synthetic construct; identical to SEQ ID NO: 6
  source 1..30: mol_type = other DNA

SEQ ID NO: 9 - DNA, 30 bp, synthetic construct; identical to SEQ ID NO: 6
  source 1..30: mol_type = other DNA

SEQ ID NO: 10 - DNA, 30 bp, synthetic construct; identical to SEQ ID NO: 6
  source 1..30: mol_type = other DNA

SEQ ID NO: 11 - DNA, 10 bp, synthetic construct
  source 1..10: mol_type = other DNA
tatttccctc

SEQ ID NO: 12 - DNA, 20 bp, synthetic construct
  source 1..20: mol_type = other DNA
ttttctctcttatttccctc

SEQ ID NO: 13 - DNA, 40 bp, synthetic construct
  source 1..40: mol_type = other DNA
tatatttcttcttactcttcttttctctcttatttccctc

SEQ ID NO: 14 - DNA, 60 bp, synthetic construct
  source 1..60: mol_type = other DNA
ttgctcaccatggtggctcttatatttcttcttactcttcttttctctcttatttccctc

SEQ ID NO: 15 - DNA, 60 bp, synthetic construct; identical to SEQ ID NO: 14
  source 1..60: mol_type = other DNA

SEQ ID NO: 16 - DNA, 60 bp, synthetic construct; identical to SEQ ID NO: 14
  source 1..60: mol_type = other DNA

SEQ ID NO: 17 - DNA, 30 bp, synthetic construct
  source 1..30: mol_type = other DNA
gcccttgctcaccatggtggctcttatatt

SEQ ID NO: 18 - DNA, 30 bp, synthetic construct; identical to SEQ ID NO: 17
  source 1..30: mol_type = other DNA

SEQ ID NO: 19 - DNA, 10 bp, synthetic construct
  source 1..10: mol_type = other DNA
accatggtgg

SEQ ID NO: 20 - DNA, 20 bp, synthetic construct
  source 1..20: mol_type = other DNA
tcaccatggtggctcttata

SEQ ID NO: 21 - DNA, 40 bp, synthetic construct
  source 1..40: mol_type = other DNA
ctcgcccttgctcaccatggtggctcttatatttcttctt

SEQ ID NO: 22 - DNA, 30 bp, synthetic construct
  source 1..30: mol_type = other DNA
cttcctactcaggctttattcaaagaccaa

SEQ ID NO: 23 - DNA, 30 bp, synthetic construct; identical to SEQ ID NO: 22
  source 1..30: mol_type = other DNA

SEQ ID NO: 24 - DNA, 30 bp, synthetic construct
  source 1..30: mol_type = other DNA
tttttttttttttttttttttttttttttt

SEQ ID NO: 25 - DNA, 52 bp, synthetic construct
  source 1..52: mol_type = other DNA
ttcttactcttcttttctctcttatttccctctttttttttttttttttttt

SEQ ID NO: 26 - DNA, 30 bp, synthetic construct; identical to SEQ ID NO: 6
  source 1..30: mol_type = other DNA

SEQ ID NO: 27 - DNA, 30 bp, synthetic construct; identical to SEQ ID NO: 6
  source 1..30: mol_type = other DNA

SEQ ID NO: 28 - DNA, 30 bp, synthetic construct; identical to SEQ ID NO: 6
  source 1..30: mol_type = other DNA

SEQ ID NO: 29 - DNA, 30 bp, synthetic construct; identical to SEQ ID NO: 6
  source 1..30: mol_type = other DNA

SEQ ID NO: 30 - DNA, 30 bp, synthetic construct; identical to SEQ ID NO: 6
  source 1..30: mol_type = other DNA

SEQ ID NO: 31 - DNA, 30 bp, synthetic construct; identical to SEQ ID NO: 6
  source 1..30: mol_type = other DNA

(19) United States
(12) Patent Application Publication
(10) Pub. No.: US 2025/0360203 A1
(43) Pub. Date: Nov. 27, 2025

(54) MRNA COMPOSITION FOR REGULATING MRNA TRANSLATION COMPRISING MRNA AND ANTISENSE OLIGONUCLEOTIDE COMPLEMENTARY TO PORTION OF THE MRNA

(71) Applicant: KOREA ADVANCED INSTITUTE OF SCIENCE AND TECHNOLOGY, Daejeon (KR)

(72) Inventors: Yong Woong JUN, Daejeon (KR); Francis Erluph Barro CABANTING, Daejeon (KR); Tae Ung JEONG, Daejeon (KR); Ju Hyung SONG, Daejeon (KR); Jihun CHOI, Daejeon (KR)

(73) Assignee: KOREA ADVANCED INSTITUTE OF SCIENCE AND TECHNOLOGY, Daejeon (KR)

(21) Appl. No.: 19/214,376

(22) Filed: May 21, 2025

(30) Foreign Application Priority Data
May 23, 2024 (KR) 10-2024-0066981

(51) Int. Cl. A61K 39/385; A61K 39/00; A61K 47/69; C12N 15/113

(52) U.S. Cl. CPC A61K 39/385; A61K 47/6911; A61K 47/6929; C12N 15/113; A61K 2039/53; C12N 2310/11; C12N 2310/335

(57) ABSTRACT

Disclosed is an mRNA composition containing: an mRNA sequence comprising, in order from 5′ to 3′, a 5′ cap region, a 5′ UTR region, a start codon region, a 3′ UTR region, and a poly (A) tail region; and an antisense oligonucleotide containing a region complementary to the 5′ cap region. The mRNA composition containing the mRNA sequence and the antisense oligonucleotide containing a region complementary to the 5′ cap region of the mRNA sequence may regulate the translation rate of the mRNA, enables selective protein expression based on the type of nucleotide modification and DNA repair mechanism, and may improve the stability of the mRNA against RNA-degrading proteins, thereby improving the stability and efficiency of mRNA vaccines or therapeutics.

### CROSS-REFERENCE TO RELATED APPLICATION

This application claims priority based on Korean Patent Application No. 10-2024-0066981 filed May 23, 2024, the contents of which are herein incorporated by reference in its entirety.

### INCORPORATION BY REFERENCE OF SEQUENCE LISTING

The instant application contains a Sequence Listing which has been filed electronically in xml format and is hereby incorporated by reference in its entirety. Said xml file, created on May 20, 2025, is named Q309402 SEQ LIST ST26.xml and is 29.2 KB in size.

### BACKGROUND OF THE INVENTION

### Field of the Invention

The present invention relates to a composition for regulating mRNA translation comprising an mRNA and an antisense oligonucleotide complementary to a portion of the mRNA, wherein the composition regulates the translation rate of the mRNA and improves the stability of the mRNA against RNA-degrading proteins.

### Description of the Related Art

mRNA vaccines are used as a novel therapeutic method that provides immunity difficult to achieve by conventional vaccines, and have played a pivotal role in protecting billions of people from pandemics, proving their effectiveness in a wide range of populations. mRNA is a substance that carries the genetic information necessary for protein expression. Based on this characteristic, an mRNA vaccine, when injected in vivo, expresses a protein that mimics the target virus, and the human body recognizes the protein as an antigen and activates the immune system through antibody formation.

Although mRNA therapeutics have advantages over existing vaccines, such as lower infectivity and lower manufacturing costs, they still have shortcomings that have not been resolved. The first shortcoming is that mRNA is a substance that is easily degraded. Due to poor temperature stability thereof, mRNA should be stored at a temperature of −60° C. or lower. Also, after injection into cells, mRNA is degraded by proteins such as RNases, and thus the time for mRNA to express a mimicking protein does not last long (Uddin, M. N.; Roni, M. A. “Challenges of storage and stability of mRNA-based COVID-19 Vaccines” Vaccines 2021, 9, 1033).

Another shortcoming is that the process of protein expression from mRNA cannot be controlled at all because the entire process of expressing the genetic information of mRNA, delivered in vivo, into a protein, relies on protein expression factors existing in the human body (Nils Klocker et. al., “Photocaged 5′cap analogues for optical control of mRNA translation in cells” Nature Chemistry, 2022, 14, 906). The inability to control the amount and rate of antigen production is a very fatal shortcoming. Since antigens are recognized as “foreign proteins” in vivo, causing autoimmune reactions, the immune response caused by an antigen that is rapidly produced is actually one of the major causes of various side effects. As a representative example, it has been reported that most people experience fever and fatigue after receiving COVID-19 mRNA vaccines, wherein this adverse effect results from the antigen-antibody immune response that temporarily and very quickly occurs, as the expression of the delivered mRNA is not controlled and the antigen is expressed simultaneously (Oleguer, Pares-Badell et. al., “Local and systemic adverse reaction to mRNA COVID-19 vaccines comparing two vaccine types and occurrence of previous COVID19 infection” Vaccines, 2021, 9, 1463).

To solve problems associated with the stability of mRNA, various technologies have been developed, such as modifying internucleotide linkages, or modifying nucleotide structures, or using a nucleotide sequence with a secondary structure. In order to increase the in vivo translation efficiency of mRNA delivered in vivo, a method of using mRNA produced by modifying the nucleotides of mRNA has been attempted, and it is known that modified nucleic acids have higher in vivo translation efficiency than mRNA based on unmodified nucleic acids.

Examples of modifications that increase the in vivo stability of mRNA by reducing the immune response thereof or increase translation of mRNA include modified nucleic acids in which uridine is modified to pseudouridine (WO 2007/024708, WO 2011/071931, etc.), modified nucleic acids in which uridine is modified to N1-methylpseudouridine (WO 2012/045075 and WO 2013/052523, etc.), and modified nucleic acids in which uridine is modified to 5-methoxyuridine (US 2020-0030460, etc.). Also, examples of technology for controlling the expression rates of proteins include technologies that express proteins using light as a medium by modifying RNA with a light-responsive compound. However, most of these technologies have disadvantages in that the production cost of mRNA is very high and in that it is impossible to control protein expression using light in vivo.

Accordingly, the present inventors have designed an antisense oligonucleotide complementary to a specific region of mRNA, particularly the 5′ cap region of the mRNA, or an antisense oligonucleotide complementary to a specific region of mRNA and containing some modified nucleotides, and have found that the antisense oligonucleotide may inhibit protein expression by interfering with the binding of an RNA-binding protein to the mRNA, and this antisense oligonucleotide may be separated from the mRNA under specific conditions, so that protein expression possible is possible again, thereby easily regulating mRNA translation. Based on this finding, the present invention has been completed.

The above information disclosed in this Background section is only for enhancement of understanding of the background of the present invention. Therefore, it may not contain information that forms a conventional art that is already known in the art to which the present invention pertains.

### SUMMARY OF THE INVENTION

An object of the present invention is to provide a composition for regulating mRNA translation comprising an mRNA and an antisense oligonucleotide complementary to a portion of the mRNA, in which the composition regulates the translation rate of the mRNA and improves the stability of mRNA against RNA-degrading proteins.

To achieve the above object, the present invention provides an mRNA composition comprising: an mRNA sequence comprising, in order from 5′ to 3′, a 5′ cap region, a 5′ UTR region, a start codon region, a 3′ UTR region, and a poly (A) tail region; and an antisense oligonucleotide complementary to the 5′ cap region.

The mRNA composition according to the present invention, which comprises the mRNA sequence and the antisense oligonucleotide complementary to the 5′ cap region of the mRNA sequence, may regulate the translation rate of the mRNA in vivo, enable selective protein expression based on the type of nucleotide modification and DNA repair mechanism, and exhibit excellent stability against RNA-degrading proteins.

### BRIEF DESCRIPTION OF THE DRAWINGS

FIG. 1 is a schematic diagram showing the interaction between mRNA and initiation factors necessary for protein expression.

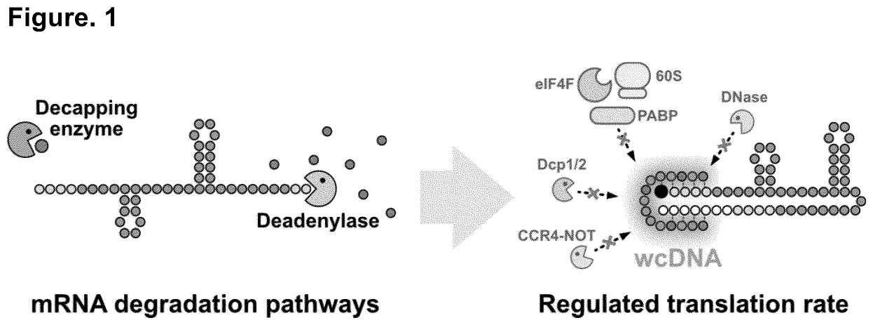

FIG. 2 is a schematic diagram showing the interaction between an antisense oligonucleotide of the present invention and mRNA.

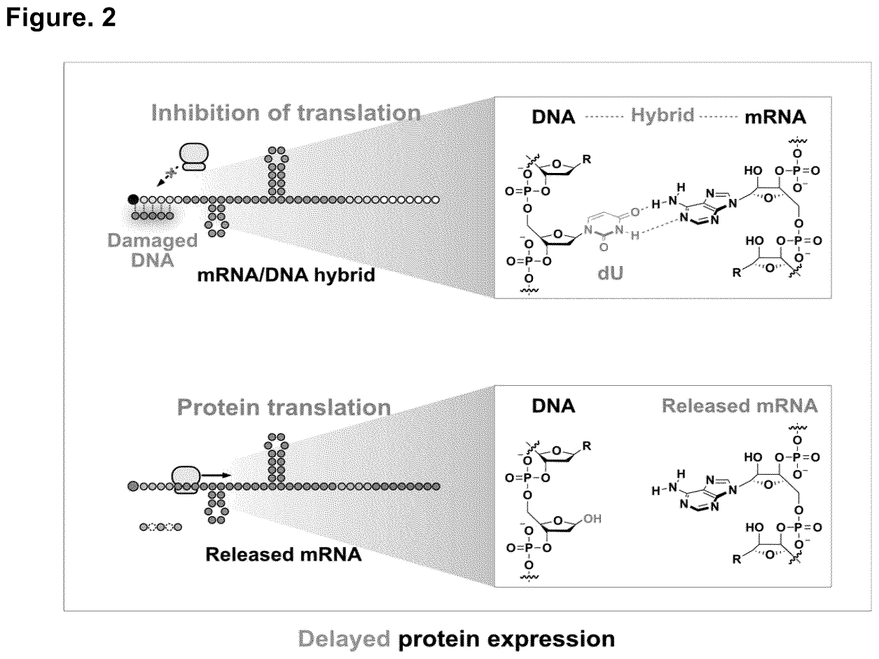

FIG. 3 depicts graphs showing results of evaluating the binding of cDNA capable of inhibiting mRNA translation to each of the 5′ cap region, 5′ UTR region, start codon region, 3′ UTR region, and poly (A) tail region of mRNA.

FIG. 4 depicts graphs showing the results of evaluating the protein expression inhibitory effect depending on the length of cDNA for the 5′ cap region of mRNA.

FIG. 5 is a graph showing the results of evaluating the restoration of mRNA translation that appears when using uracil-containing cDNA and UDG.

FIG. 6 is a graph showing that the time for uracil-containing CDNA to be repaired may be controlled depending on the repair activity of UDG by adjusting the concentration of UDG.

FIG. 7 is a graph showing that mRNA translation is regulated by controlling the number of uracils contained in CDNA.

FIG. 8 is a graph showing that mRNA translation is regulated by controlling the nucleotide length of cDNA.

FIG. 9 is a graph showing that mRNA translation is regulated by controlling the nucleotide length of cDNA.

FIG. 10 is a graph showing the correlation between the distance between dUs and the mRNA translation restoration rate for cDNA modified with a 2′-methoxy group introduced to stabilize the 5′ cap region of mRNA as a therapeutic agent and for cDNA with both sugar and phosphate modifications.

FIG. 11 is a schematic diagram showing the structure and mechanism of DNA that binds to both the 5′ cap region and poly (A) tail region of mRNA.

FIG. 12 shows the stability-enhancing effect of cDNA that binds to both the 5′ cap region and poly (A) tail region of mRNA.

### DETAILED DESCRIPTION OF THE INVENTION

Unless otherwise defined, all technical and scientific terms used in the present specification have the same meanings as commonly understood by those skilled in the art to which the present invention pertains. In general, the nomenclature used in the present specification is well known and commonly used in the art.

In order to solve the problems of mRNA vaccines and therapeutics that mRNA is easily degraded and it is difficult to control the process of protein expression from mRNA as the in vivo translation of mRNA relies on protein expression factors existing in vivo, the present inventors have developed an antisense oligonucleotide complementary to each of the 5′ cap region, start codon region, 3′ UTR region, and poly (A) tail region of mRNA and containing some modified nucleotides. The present inventors have found that a composition comprising mRNA and an antisense oligonucleotide complementary to a portion of the mRNA and containing some modified nucleotides may control the translation rate of the mRNA depending on the activity of the DNA repair mechanism by controlling the type or number of nucleotide modifications of cDNA based on the DNA repair mechanism at the target position, and that the cDNA containing modified nucleotides binds to regions that are easily degraded by RNA-degrading proteins, such as a 5′ cap region and a poly (A) tail region, thereby protecting the mRNA from the RNA-degrading proteins, thus increasing the stability of the mRNA.

Therefore, in one aspect, the present invention relates to an mRNA composition comprising: an mRNA sequence comprising, in order from 5′ to 3′, a 5′ cap region, a 5′ UTR region, a start codon region, a 3′ UTR region, and a poly (A) tail region; and an antisense oligonucleotide containing a region complementary to the 5′ cap region.

In the present invention, the mRNA structure may comprise, in order from 5′ to 3′, a 5′ cap region, a 5′ UTR region, a start codon region, a 3′ UTR region, and a poly (A) tail region. The term “mRNA structure” is used with the same meaning as the term “nucleic acid molecule” or “mRNA molecule”, and means a form of a structure that includes an mRNA encoding a target coding region and is administered in vivo for expression of the target coding region.

In the present invention, the mRNA structure may comprise the nucleotide sequence of SEQ ID NO: 1, without being limited thereto.

In the present invention, the antisense oligonucleotide may be complementary DNA (CDNA), and may be cDNA in which at least one nucleotide is modified. Preferably, the antisense oligonucleotide may be cDNA in which at least one thymine is substituted with uracil, without being limited thereto.

The 5′ cap of native mRNA is involved in nuclear export, increasing mRNA stability, and binds to the mRNA Cap Binding Protein (CBP), which is responsible for mRNA stability in the cell and translation competency through the association of CBP with poly (A) binding protein to form the mature cyclic mRNA species. The cap further assists the removal of 5′ proximal introns during mRNA splicing.

In the present invention, the 5′ cap is typically a modified nucleotide (cap analogue), particularly a guanine nucleotide added to the 5′ end of an mRNA molecule. Examples of the 5′ cap include, but are not limited to, glyceryl, inverted deoxy abasic residue (moiety), 4′, 5′ methylene nucleotide, 1-(beta-D-erythrofuranosyl) nucleotide, 4′-thio nucleotide, carbocyclic nucleotide, 1, 5-anhydrohexitol nucleotide, L-nucleotides, alpha-nucleotide, modified base nucleotide, threo-pentofuranosyl nucleotide, acyclic 3′, 4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1, 4-butanediol phosphate, 3′-phosphoramidate, hexyl phosphate, aminohexyl phosphate, 3′-phosphate, 3′ phosphorothioate, phosphorodithioate, or bridging or non-bridging methylphosphonate moiety.

In the present invention, the 5′ cap region may comprise the nucleotide sequence of SEQ ID NO: 2, without being limited thereto.

In the present invention, “UTR” refers to an “untranslated region” located upstream (5′) and/or downstream (3′) a coding region of a nucleic acid molecule as described herein, thereby typically flanking said coding region.

Accordingly, the term “UTR” generally encompasses 3′-untranslated regions (“3′-UTRs”) and 5′-untranslated regions (“5′-UTRS”). UTRs may typically comprise or consist of nucleic acid sequences that are not translated into protein. Typically, UTRs comprise “regulatory elements”.

The term “regulatory element” refers to a nucleic acid sequence having gene regulatory activity, the ability to affect the expression, in particular transcription or translation, of an operably (in cis or trans) linked transcribable nucleic acid sequence. The term encompasses promoters, enhancers, internal ribosomal entry sites (IRES), introns, leaders, transcription termination signals, such as polyadenylation signals and poly-U sequences and other expression control elements. Regulatory elements may act constitutively or in a time-and/or cell specific manner.

Optionally, regulatory elements may exert their function via interacting with (e.g., recruiting and binding) of regulatory proteins capable of modulating (inducing, enhancing, reducing, abrogating, or preventing) the expression, in particular transcription of a gene.

UTRs are preferably “operably linked”, i.e. placed in a functional relationship, to a coding region, preferably in a manner that allows control (i.e., modulation or regulation, preferably enhancement) over the expression of said coding sequence.

In the present invention, the term “5′-UTR” refers to a part of a nucleic acid molecule, which is located 5′ (i.e., “upstream”) of an open reading frame and which is not translated into protein. In the context of the present invention, a 5′-UTR starts with the transcriptional start site and ends one nucleotide before the start codon of the open reading frame.

The 5′-UTR may comprise elements for regulating gene expression, also called “regulatory elements”. Such regulatory elements may be, for example, ribosomal binding sites. The 5′-UTR may be post-transcriptionally modified, for example by addition of a 5′-cap. Thus, 5′-UTRs may preferably correspond to the sequence of a nucleic acid, in particular a mature mRNA, which is located between the 5′-cap and the start codon, and more specifically to a sequence, which extends from a nucleotide located 3′ to the 5′-cap, preferably from the nucleotide located immediately 3′ to the 5′-cap, to a nucleotide located 5′ to the start codon of the protein coding sequence (transcriptional start site), preferably to the nucleotide located immediately 5′ to the start codon of the protein coding sequence (transcriptional start site).

In the present invention, the 3′ UTR region may comprise the nucleotide sequence of SEQ ID NO: 4, without being limited thereto.

In the present invention, the mRNA structure may comprise a region encoding a signal peptide between the 5′ UTR region and the start codon region, i.e., a polynucleotide encoding the signal peptide.

In the present invention, the start codon region may comprise the nucleotide sequence of SEQ ID NO: 3, without being limited thereto.

The signal peptide may be derived from an antigenic polypeptide, immunoglobulin E (IgE), or tissue plasminogen activator (tPA), without being limited thereto. The polynucleotide encoding the signal peptide may be codon-optimized.

In the present invention, the mRNA structure may comprise at least one coding region between the 5′ UTR region and the 3′ UTR region.

In the present invention, the coding region may encode any one or more proteins selected from the group consisting of an antigenic protein, an allergenic protein, a therapeutic protein, and a fragment, variant or derivative of the protein.

In the present invention, the region encoding the protein, etc., i.e., the polynucleotide encoding the protein, is used, without being limited thereto.

For example, the antigenic protein may be any one or more selected from the group consisting of a tumor antigen, a pathogenic antigen, an autoantigen, an alloantigen, and an allergic antigen, without being limited thereto.

In the present invention, the term “tumor antigen” refers to antigenic (poly-) peptides or proteins derived from or associated with a (preferably malignant) tumor or a cancer disease. As used herein, the terms “cancer” and “tumor” are used interchangeably to refer to a neoplasm characterized by the uncontrolled and usually rapid proliferation of cells that tend to invade surrounding tissue and to metastasize to distant body sites. The term encompasses benign and malignant neoplasms. Malignancy in cancers is typically characterized by anaplasia, invasiveness, and metastasis; whereas benign malignancies typically have none of those properties. The terms “cancer” and “tumor” in particular refer to neoplasms characterized by tumor growth, but also to cancers of the blood and the lymphatic system. A “tumor antigen” is typically derived from a tumor/cancer cell, preferably a mammalian tumor/cancer cell, and may be located in or on the surface of a tumor cell derived from a mammalian, preferably from a human, tumor, such as a systemic or a solid tumor. “Tumor antigens” generally include tumor-specific antigens (TSAs) and tumor-associated-antigens (TAAs). TSAs typically result from a tumor specific mutation and are specifically expressed by tumor cells. TAAs, which are more common, are usually presented by both tumor and “normal” (healthy, non-tumor) cells.

In the present invention, the tumor antigen may be selected from the group consisting of NYESO-1, HER-2/neu, MAGE-1, Tyrosinase, MUC1, CEA, Mam-A, hTERT, Syalyl-Tn, WT1, alpha-fetoprotein, CA-125, gp-100, p53, Ras, Src, EGFRVIII, PSMA, GD2, Bcr-abl, Survivin, PSA, EphA2, PAP, AFP, EpCAM, ALK, Mesothelin, PSCA, MART-1, Melan-A, SCP-1, SPAG9, AKAP4, and OY-TES-1, without being limited thereto.

In the present invention, the pathogenic antigen may be selected from the group consisting of bacterial, viral, fungal and protozoal antigens.

In the present invention, the pathogenic antigen may be derived from Influenza virus, respiratory syncytial virus (RSV), coronavirus, Herpes Simplex Virus (HSV), Human Papilloma Virus (HPV), Human Immunodeficiency Virus (HIV), Plasmodium, Staphylococcus aureus, Dengue virus, Chlamydia trachomatis, Cytomegalovirus (CMV), Hepatitis B Virus (HBV), Mycobacterium tuberculosis, Rabies virus, and Yellow Fever Virus, or an isoform, homolog, fragment, variant or derivative of any of these proteins.

In the present invention, the antigenic polypeptide or the immunogenic protein thereof may be an influenza virus antigenic polypeptide, and may be at least one selected from the group consisting of the defined antigenic subdomains of hemagglutinin (HA), termed HA1, HA2, or a combination of HA1 and HA2, and neuraminidase (NA), nucleoprotein (NP), matrix protein 1 (M1), matrix protein 2 (M2), non-structural protein 1 (NS1) and non-structural protein 2 (NS2), without being limited thereto.

In the present invention, the influenza antigenic polypeptide or the immunogenic protein thereof may be derived from an influenza virus strain selected from the group consisting of influenza B Yamagata, influenza B Victoria, and influenza A H3N2, and more preferably, an HA protein derived from each strain, without being limited thereto.

In the present invention, the polynucleotide encoding the influenza antigenic polypeptide or the immunogenic protein thereof may be codon-optimized.

In the present invention, the mRNA construct may further comprise a poly (A) tail or a poly (A) tail-like sequence. In a further embodiment, terminal groups on the poly (A) tail may be incorporated for stabilization. In another embodiment, the poly (A) tail comprises des-3′ hydroxyl tails.

During RNA processing, a long chain of adenine nucleotides (poly (A) tail) may be added to a polynucleotide such as an mRNA molecule in order to increase stability. Immediately after transcription, the 3′ end of the transcript may be cleaved to free a 3′ hydroxyl. Then, poly-A polymerase adds a chain of adenine nucleotides to the RNA. The process, called polyadenylation, adds a poly-A tail that can be between, for example, approximately 80 to approximately 250 residues long (including approximately 80, 90, 100, 110, 120, 130, 140, 150, 160, 170, 180, 190, 200, 210, 220, 230, 240 or 250 residues long). PolyA tails may also be added after the construct is exported from the nucleus.

Unique poly-A tail lengths provide certain advantages to the polynucleotides of the present invention. Generally, the length of a poly-A tail, when present, is greater than 30 nucleotides in length. In another embodiment, the poly-A tail is greater than 35 nucleotides in length (e.g., at least or greater than about 35, 40, 45, 50, 55, 60, 70, 80, 90, 100, 120, 140, 160, 180, 200, 250, 300, 350, 400, 450, 500, 600, 700, 800, 900, 1,000, 1,100, 1,200, 1,300, 1,400, 1,500, 1,600, 1,700, 1,800, 1,900, 2,000, 2,500, and 3,000 nucleotides).

Additionally, multiple distinct polynucleotides may be linked together via the PABP (poly (A) binding protein) through the 3′-end using modified nucleotides at the 3′-terminus of the poly (A) tail. Transfection experiments may be conducted in relevant cell lines and protein production may be assayed by ELISA at 12 hr, 24 hr, 48 hr, 72 hr and day 7 post-transfection.

In the present invention, the poly (A) tail region may comprise the nucleotide sequence of SEQ ID NO: 5, without being limited thereto.

In the present invention, the mRNA structure may comprise one or more backbone-modified, sugar-modified, or base-modified nucleic acids.

### Backbone Modifications

The phosphate backbone may further be modified in the modified nucleosides and nucleotides, which may be incorporated into a modified RNA molecule including an mRNA sequence as described herein. The phosphate groups of the backbone may be modified by replacing one or more of the oxygen atoms with a different substituent. Further, the modified nucleosides and nucleotides may include the full replacement of an unmodified phosphate moiety with a modified phosphate as described herein. Examples of modified phosphate groups include, but are not limited to, phosphorothioate, phosphoroselenates, boranophosphates, boranophosphate esters, hydrogen phosphonates, phosphoroamidates, alkyl or aryl phosphonates and phosphotriesters. Phosphorodithioates have both non-linking oxygens substituted by sulfur.

The phosphate linker may also be modified by the replacement of a linking oxygen with nitrogen (bridged phosphoroamidates), sulfur (bridged phosphorothioates) and carbon (bridged methylene-phosphonates), without being limited thereto.

### Sugar Modifications

The modified nucleosides and nucleotides, which may be incorporated into a modified RNA molecule as described herein, may be modified in the sugar moiety. For example, the 2′ hydroxyl group (OH) may be modified or substituted with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH₂CH₂O)nCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, may be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group may be attached to the sugar through a linker, wherein the linker comprises one or more of the atoms C, N, and O, without being limited thereto.

The sugar group may also contain one or more carbons that possess the opposite stereochemical configuration than that of the corresponding carbon in ribose. Thus, a modified

RNA molecule may include nucleotides containing, for instance, arabinose as the sugar, without being limited thereto.

### Base Modifications

The modified nucleosides and nucleotides, which may be incorporated into a modified mRNA compound including an mRNA sequence as described herein, may further be modified in the nucleobase moiety. Examples of nucleobases found in mRNA include, but are not limited to, adenine, guanine, cytosine and uracil. For example, the nucleosides and nucleotides described herein may be chemically modified on the major groove face. In some embodiments, the major groove chemical modifications may include an amino group, a thiol group, an alkyl group, or a halo group.

In some embodiments, the nucleotide analogues/modifications are selected from base modifications, which are selected from 2-amino-6-chloropurineriboside-5′-triphosphate, 2-aminopurine-riboside-5′-triphosphate; 2-aminoadenosine-5′-triphosphate, 2′-amino-2′-deoxycytidine-triphosphate, 2-thiocytidine-5′-triphosphate, 2-thiouridine-5′-triphosphate, 2′-fluorothymidine-5′-triphosphate, 2′-O-methyl inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-bromo-2′-deoxycytidine-5′-triphosphate, 5-bromo-2′-deoxyuridine-5′-triphosphate, 5-iodocytidine-5′-triphosphate, 5-iodo-2′-deoxycytidine-5′-triphosphate, 5-iodouridine-5′-triphosphate, 5-iodo-2′-deoxyuridine-5′-triphosphate, 5-methylcytidine-5′-triphosphate, 5-methyluridine-5′-triphosphate, 5-propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, O6-methylguanosine-5′-triphosphate, pseudouridine-5′-triphosphate, or puromycin-5′-triphosphate, xanthosine-5′-triphosphate, without being limited thereto.

In some embodiments, modified nucleosides include, but are not limited to, pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include, but are not limited to, 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-4-thio-1-methyl-1-deaza-pseudoisocytidine, pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In some embodiments, modified nucleosides include, but are not limited to, 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2, 6-diaminopurine, 7-deaza-8-aza-2, 6-diaminopurine, 1-methyladenosine, N6-methyladenosine, N6-isopentenyladenosine, N6-(cis-2-methylthio-N6-(cis-hydroxyisopentenyl) adenosine, hydroxyisopentenyl) adenosine, N6-glycinylcarbamoyladenosine, N6-threonylcarbamoyladenosine, 2-methylthio-N6-threonyl carbamoyladenosine, N6, N6-dimethyladenosine, 7-methyladenine, 2-methylthio-adenine, and 2-methoxy-adenine.

In some embodiments, modified nucleosides include, but are not limited to, inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2, N2-dimethylguanosine, 8-oxo-guanosine, 7-methyl-8-oxo-guanosine, 1-methyl-6-thio-guanosine, N2-methyl-6-thio-guanosine, and N2, N2-dimethyl-6-thio-guanosine.

In some embodiments, the nucleotide may be modified on the major groove face and may include replacing hydrogen on C-5 of uracil with a methyl group or a halo group, and a modified nucleoside may be 5′-O-(1-thiophosphate)-adenosine, 5′-O-(1-thiophosphate)-cytidine, 5′-O-(1-thiophosphate)-guanosine, 5′-O-(1-thiophosphate)-uridine or 5′-O-(1-thiophosphate)-pseudouridine, without being limited thereto.

In some embodiments, a modified mRNA may comprise nucleoside modifications selected from 6-aza-cytidine, 2-thio-cytidine, a-thio-cytidine, pseudo-iso-cytidine, 5-aminoallyl-uridine, 5-iodo-uridine, N1-methyl-pseudouridine, 5, 6-dihydrouridine, x-thio-uridine, 4-thio-uridine, 6-aza-uridine, 5-hydroxy-uridine, deoxy-thymidine, 5-methyl-uridine, pyrrolo-cytidine, inosine, a-thio-guanosine, 6-methyl-guanosine, 5-methyl-cytidine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, x-thio-adenosine, 8-azido-adenosine, and 7-deaza-adenosine, without being limited thereto.

In another aspect, the present invention relates to a pharmaceutical composition for preventing or treating a disease selected from the group consisting of cancer, a tumor, an autoimmune disease, a genetic disease, an inflammatory disease, a viral infection and a bacterial infection, the pharmaceutical composition containing, as an active ingredient, the composition comprising the mRNA structure and the antisense oligonucleotide.

As used herein, the term “prevention” or “preventing” refers to any action of suppressing the above-described disease or delaying the progression of the disease by administering the pharmaceutical composition according to the present invention.

As used herein, the term “treatment” or “treating” refers to any action of alleviating or beneficially changing the symptoms of the above-described disease by administering the pharmaceutical composition according to the present invention.

The pharmaceutical composition of the present invention may contain the active ingredient in combination with one or more pharmaceutically or physiologically acceptable carriers, diluents or excipients. The pharmaceutical composition may contain a buffer, such as neutral buffered saline, phosphate buffered saline, citrate buffered solution, or the like; a carbohydrate, such as glucose, mannose, sucrose, dextran, or mannitol; a protein; a polypeptide or an amino acid, such as glycine; an antioxidant; a chelating agent, such as EDTA or glutathione; an adjuvant (e.g., aluminum hydroxide); and a preservative.

The pharmaceutical composition of the present invention can be administered orally or parenterally. For example, it may be administered intravenously, subcutaneously, intradermally, intramuscularly, intraperitoneally, intratumorally, intracerebrally, intracranially, intrapulmonary, or intrarectally, without being limited thereto.

The pharmaceutical composition of the present invention is administered in a pharmaceutically effective amount.

As used herein, the term “pharmaceutically effective amount” refers to an amount sufficient to treat a disease at a reasonable benefit/risk ratio applicable to any medical treatment. The effective amount may be determined depending on factors, including the kind and severity of the patient's disease, the activity of the drug, sensitivity to the drug, the time of administration, the route of administration, excretion rate, and concomitantly used drugs, as well as other factors well known in the medical field.

The pharmaceutical composition of the present invention may be administered individually or in combination with other therapeutic agents, and may be administered sequentially or simultaneously with conventional therapeutic agents. The pharmaceutical composition may be administered in a single or multiple dosage form. It is important to administer the pharmaceutical composition in the minimum amount that can exhibit the maximum effect without causing side effects, in view of all the above-described factors, and this amount can be easily determined by a person skilled in the art.

Specifically, the effective amount of the pharmaceutical composition of the present invention may vary depending on the patient's age, sex, condition, and body weight, the absorption rate, inactivation rate and excretion rate of the active ingredient in the body, the type of disease, and concomitantly administered drugs. Since the pharmaceutical composition of the present invention contains the above-described composition as an effective ingredient, description of overlapping contents omitted to avoid excessive complexity of the specification.

In still another aspect, the present invention relates to a method for preventing or treating cancer, a tumor, an autoimmune disease, an inflammatory disease, a viral infection, or a bacterial infection, the method comprising administering a composition of the present invention to a patient in need of prevention or treatment of cancer, a tumor, an autoimmune disease, an inflammatory disease, a viral infection, or a bacterial infection.

The dose of the mRNA may be 1 to 5 μg, 5 to 10 μg, 10 to 15 μg, 15 to 20 μg, 10 to 25 μg, 20 to 25 μg, 20 to 50 μg, 30 to 50 μg, 40 to 50 μg, 40 to 60 μg, 60 to 80 μg, 60 to 100 μg, 50 to 100 μg, 80 to 120 μg, 40 to 120 μg, 40 to 150 μg, 50 to 150 μg, 50 to 200 μg, 80 to 200 μg, 100 to 200 μg, 120 to 250 μg, 150 to 250 μg, 180 to 280 μg, 200 to 300 μg, 50 to 300 μg, 80 to 300 μg, 100 to 300 μg, 40 to 300 μg, 50 to 350 μg, 100 to 350 μg, 200 to 350 μg, 300 to 350 μg, 320 to 400 μg, 40 to 380 μg, 40 to 100 μg, 100 to 400 μg, 200 to 400 μg, 300 to 400 μg, 30 to 500 μg, 40 to 500 μg, 50 to 500 μg, 100 to 500 μg, 200 to 500 μg, 300 to 500 μg, 400 to 500 μg, 40 to 600 μg, 100 to 600 μg, 200 to 600 μg, 400to 600 μg, 40 to 700 μg, 100 to 700 μg, 300 to 700 μg, 500 to 700 μg, 40 to 800 μg, 100 to 800 μg, 200 to 800 μg, 400 to 800 μg, or 600 to 800 μg, without being limited thereto.

In yet another aspect, the present invention relates to a vaccine composition comprising the mRNA structure and the antisense oligonucleotide.

In the present invention, the “vaccine” is typically understood to be a prophylactic or therapeutic material providing at least one antigen, preferably an antigenic peptide or protein. “Providing at least on antigen” means, for example, that the vaccine comprises the antigen or that the vaccine comprises a molecule that, e.g., codes for the antigen. Accordingly, it is particularly envisaged herein that the vaccine of the present invention comprises at least one artificial nucleic acid (RNA) molecule encoding at least one antigenic (poly-) peptide or protein as defined herein, which may, for instance, be derived from a tumor antigen, a bacterial, viral, fungal or protozoal antigen, an autoantigen, an allergen, or an allogenic antigen, and preferably induces an immune response toward the respective antigen when it is expressed and presented to the immune system.

In the present invention, the mRNA structure of the vaccine composition may be complexed with one or more lipids to form lipid nanoparticles or liposomes. The lipid nanoparticles may comprise a cationic lipid, a PEG-modified lipid, a sterol, and a non-cationic lipid, without being limited thereto.

In the present invention, the mRNA structure may be complexed or associated with one or more (poly-) cationic compounds, preferably with (poly-) cationic polymers, (poly-) cationic peptides or proteins, e.g., protamine, (poly-) cationic polysaccharides and/or (poly-) cationic lipids. In this context, the term “complexed” or “associated” refers to the essentially stable combination of at least one artificial nucleic acid (RNA) molecule with one or more of the aforementioned compounds into larger complexes or assemblies without covalent binding.

### Lipids

The mRNA structure of the present invention may be complexed or associated with lipids (in particular cationic and/or neutral lipids) to form one or more lipid nanoparticles. Thus, in some embodiments, the mRNA molecules of the present invention may be provided in the form of lipid-based formulations, liposomes comprising the mRNA molecules, and/or lipid nanoparticles.

### Lipid Nanoparticles

The mRNA structure of the present invention may be complexed or associated with lipids (in particular cationic and/or neutral lipids) to form one or more lipid nanoparticles.

Lipid nanoparticles (LNPs) may comprise: (a) at least one mRNA structure of the present invention, (b) a cationic lipid, (c) an aggregation reducing agent (such as polyethylene glycol (PEG) lipid or PEG-modified lipid), (d) optionally, a non-cationic lipid (such as a neutral lipid), and (e) optionally, a sterol. In some embodiments, LNPs may comprise, in addition to the at least one mRNA structure of the present invention, (i) at least one cationic lipid; (ii) a neutral lipid; (iii) a sterol, e.g., cholesterol; and (iv) a PEG-lipid, in a molar ratio of about 20 to 60% cationic lipid: 5 to 25% neutral lipid: 25 to 55% sterol: 0.5 to 15% PEG-lipid.

### Liposomes

In some embodiments, the mRNA structure of the present invention may be formulated as liposomes. Cationic lipid-based liposomes are able to complex with negatively charged nucleic acids (e.g. RNAs) via electrostatic interactions, resulting in complexes that offer biocompatibility, low toxicity, and the possibility of the large-scale production required for in vivo clinical applications. Liposomes can fuse with the plasma membrane for uptake; once inside the cell, the liposomes are processed via the endocytic pathway and the nucleic acid is then released from the endosome/carrier into the cytoplasm. Liposomes have long been perceived as drug delivery vehicles because of their superior biocompatibility, given that liposomes are basically analogs of biological membranes, and can be prepared from both natural and synthetic phospholipids.

Liposomes typically consist of a lipid bilayer that can be composed of cationic, anionic, or neutral (phospho) lipids and cholesterol, which encloses an aqueous core. Both the lipid bilayer and the aqueous space can incorporate hydrophobic or hydrophilic compounds, respectively. Liposomes may have one or more lipid membranes. Liposomes can be single-layered, referred to as unilamellar, or multilayered, referred to as multilamellar.

Liposome characteristics and behavior in vivo can be modified by addition of a hydrophilic polymer coating, e.g. polyethylene glycol (PEG), to the liposome surface to confer steric stabilization. Furthermore, liposomes can be used for specific targeting by attaching ligands (e.g., antibodies, peptides, and carbohydrates) to their surface or to the terminal end of the attached PEG chains.

Liposomes are typically present as spherical vesicles and can range in size from 20 nm to a few microns. Liposomes can be of different sizes such as, but not limited to, a multilamellar vesicle (MLV) which may be hundreds of nanometers in diameter and may contain a series of concentric bilayers separated by narrow aqueous compartments, a small unicellular vesicle (SUV) which may be smaller than 50 nm in diameter, and a large unilamellar vesicle (LUV) which may be between 50 and 500 nm in diameter. Liposome design may include, but is not limited to, opsonins or ligands in order to improve the attachment of liposomes to unhealthy tissue or to activate events such as, but not limited to, endocytosis. Liposomes may have a low or high pH in order to improve the delivery of the pharmaceutical formulation.

In the present invention, the vaccine composition may further comprise at least one adjuvant or active agent.

An “adjuvant” or “adjuvant component” in the broadest sense is typically a pharmacological and/or immunological agent that may modify, e.g., enhance, the effect of other active agents, e.g., therapeutic agents or vaccines. In this context, an “adjuvant” may be understood as any compound, which is suitable to support administration and delivery of the vaccine composition of the present invention. Specifically, an adjuvant preferably enhances the immunostimulatory properties of the composition or vaccine to which it is added. Furthermore, such adjuvants may, without being limited thereto, initiate or increase an immune response of the innate immune system, i.e., a non-specific immune response.

“Adjuvants” typically do not elicit an adaptive immune response. Until now, “adjuvants” do not qualify as antigens. In other words, when administered, the vaccine of the present invention typically initiates an adaptive immune response due to an antigenic peptide or protein, which is encoded by the at least one coding sequence of the artificial nucleic acid (RNA) molecule contained in said vaccine.

Suitable adjuvants may be selected from any adjuvant known to a skilled person and suitable for the present case, i.e., supporting the induction of an immune response in a mammal, and include, but are not limited to, TDM, MDP, muramyl dipeptide, pluronics, alum solution, aluminum hydroxide, ADJUMER™ (polyphosphazene); aluminum phosphate gel; glucans from algae; Algammulin; aluminum hydroxide gel (alum); highly protein-absorbing aluminum hydroxide gel; low viscosity aluminum hydroxide gel; AF or SPT (emulsion of squalane (5%), Tween 80 (0.2%), Pluronic L121 (1.25%), phosphate-buffered saline, pH 7.4); AVRIDINE™ (propanediamine); a group of substances that correspond to pathogen-associated molecular patterns (PAMPs) and respond to pattern recognition receptors (PRRs); CpG DNA; lipoprotein; flagella; poly I:C; saponin; squalene; tricaprin; 3D-MPL; or detoxified lipooligosaccharide (dLOS).

Hereinafter, the present invention will be described in more detail by way of examples. These examples are only intended to explain the present invention in more detail, and it will be apparent to those skilled in the art that the scope of the present invention according to the subject matter of the present invention is not limited by these examples.

### EXAMPLE 1

### mRNA Structure

The sequence of eGFP mRNA used to confirm protein expression regulation is shown in Table 1 below.

TABLE 1

| SEQ ID | | |
|---|---|---|
| NO. | Name | Sequence |
| 1 | eGFP mRNA | GAGGGAAAUAAGAGAGAAAAGAAGAGUAAGAAGAAAUAUAAGAGCCACCAU |
| | | GGUGAGCAAGGGCGAGGAGCUGUUCACCGGGGUGGUGCCCAUCCUGGUCGA |
| | | GCUGGACGGCGACGUAAACGGCCACAAGUUCAGCGUGUCCGGCGAGGGCGA |
| | | GGGCGAUGCCACCUACGGCAAGCUGACCCUGAAGUUCAUCUGCACCACCGG |
| | | CAAGCUGCCCGUGCCCUGGCCCACCCUCGUGACCACCCUGACCUACGGCGU |
| | | GCAGUGCUUCAGCCGCUACCCCGACCACAUGAAGCAGCACGACUUCUUCAA |
| | | GUCCGCCAUGCCCGAAGGCUACGUCCAGGAGCGCACCAUCUUCUUCAAGGA |
| | | CGACGGCAACUACAAGACCCGCGCCGAGGUGAAGUUCGAGGGCGACACCCU |
| | | GGUGAACCGCAUCGAGCUGAAGGGCAUCGACUUCAAGGAGGACGGCAACAU |
| | | CCUGGGGCACAAGCUGGAGUACAACUACAACAGCCACAACGUCUAUAUCAU |
| | | GGCCGACAAGCAGAAGAACGGCAUCAAGGUGAACUUCAAGAUCCGCCACAA |
| | | CAUCGAGGACGGCAGCGUGCAGCUCGCCGACCACUACCAGCAGAACACCCC |
| | | CAUCGGCGACGGCCCCGUGCUGCUGCCCGACAACCACUACCUGAGCACCCA |
| | | GUCCGCCCUGAGCAAAGACCCCAACGAGAAGCGCGAUCACAUGGUCCUGCU |
| | | GGAGUUCGUGACCGCCGCCGGGAUCACUCUCGGCAUGGACGAGCUGUACAA |
| | | GUAAGCGGCCGCUUAAUUAAGCUGCCUUCUGCGGGGCUUGCCUUCUGGCCA |
| | | UGCCCUUCUUCUCUCCCUUGCACCUGUACCUCUUGGUCUUUGAAUAAAGCC |
| | | UGAGUAGGAAGAAAAAAAAAAAAAAAAAAAAAAAAAAAAAAAAAAAAAAAA |
| | | AAAAAAAAAAAAAAAAAAAAAAAAAAAAAAAAAAAAAAAAAAAAAAAAAAA |
| | | AAAAAAAAAAAAAAAAAAAAAAAAAAAAA |
| 2 | 5′ cap of | GAGGGAAAUAAGAGAGAAAAGAAGAGUAAG |
| | eGFP mRNA | |
| 3 | Start codon | AAUAUAAGAGCCACCAUGGUGAGCAAGGGC |
| | of eGFP mRNA | |
| 4 | 3′ UTR of | UUGGUCUUUGAAUAAAGCCUGAGUAGGAAG |
| | eGFP mRNA | |
| 5 | 3′ poly(A) | AAAAAAAAAAAAAAAAAAAAAAAAAAAAAAAAAAAAAAAAAAAAAAAAAAA |
| | tail of eGFP | AAAAAAAAAAAAAAAAAAAAAAAAAAAAAAAAAAAAAAAAAAAAAAAAAAA |
| | mRNA | AAAAAAAAAAAAAAAAAA |

### EXAMPLE 2

### Construction of mRNA Structure-Targeting DNAs

The sequences of cDNAs constructed to complementarily bind to the 5′ cap region of eGFP mRNA are shown in Table 2 below.

TABLE 2

| SEQ ID | | |
|---|---|---|
| NO. | Name | Sequence |
| 6 | 5′ cap cDNA | CTTACTCTTCTTTTCTCTCTTATTTCCCTC |
| | (30 nt) | |
| 7 | 5′ cap cDNA | CTTACTCUTCTTTTCUCTCTTATUTCCCTC |
| | (30 nt 3 dU) | |
| 8 | 5′ cap cDNA | CTTACUCTTCTUTTCTCUCTTATUTCCCTC |
| | (30 nt 4 dU) | |
| 9 | 5′ cap cDNA | CTUACTCUTCTTUTCTCTCUTATTUCCCTC |
| | (30 nt 5 dU) | |
| 10 | 5′ cap cDNA | CTUACUCTTCUTTTCUCTCTUATTUCCCUC |
| | (30 nt 7 dU) | |
| 11 | 5′ cap cDNA | TATTTCCCTC |
| | (10 nt) | |
| 12 | 5′ cap cDNA | TTTTCTCTCTTATTTCCCTC |
| | (20 nt) | |
| 13 | 5′ cap cDNA | TATATTTCTTCTTACTCTTCTTTTCTCTCTTATTTCCCTC |
| | (40 nt) | |
| 14 | 5′ cap cDNA | TTGCTCACCATGGTGGCTCTTATATTTCTTCTTACTCTTCTTTTCTCTCTT |
| | (60 nt) | ATTTCCCTC |
| 15 | 5′ cap cDNA | TTGCUCACCAUGGTGGCUCTTATATTUCTTCTTACUCTTCTTTTCUCTCTT |
| | (60 nt 7 dU) | ATTUCCCTC |
| 16 | 5′ cap cDNA | TTGCUCACCAUGGUGGCUCTTAUATTUCTTCUTACUCTTCUTTTCUCTCTU |
| | (60 nt 13 dU) | ATTUCCCUC |

The sequences of cDNAs constructed to complementarily bind to the start codon region of eGFP mRNA are shown in Table 3 below.

TABLE 3

| SEQ ID | | |
|---|---|---|
| NO. | Name | Sequence |
| 17 | Start codon | GCCCTTGCTCACCATGGTGGCTCTTATATT |
| | cDNA (30 nt) | |
| 18 | Start codon | GCCCUTGCUCACCAUGGUGGCUCTTAUATT |
| | cDNA (30 nt | |
| | 6 dU) | |
| 19 | Start codon | ACCATGGTGG |
| | cDNA (10 nt) | |
| 20 | Start codon | TCACCATGGTGGCTCTTATA |
| | cDNA (20 nt) | |
| 21 | Start codon | CTCGCCCTTGCTCACCATGGTGGCTCTTATATTTCTTCTT |
| | cDNA (40 nt) | |

The sequences of CDNAs constructed to complementarily bind to the 3′ UTR region of eGFP mRNA are shown in Table 4 below.

TABLE 4

| SEQ ID | | |
|---|---|---|
| NO. | Name | Sequence |
| 22 | 3′ UTR cDNA | CTTCCTACTCAGGCTTTATTCAAAGACCAA |
| | (30 nt) | |
| 23 | 3′ UTR cDNA | CTUCCTACUCAGGCUTTATUCAAAGACCAA |
| | (30 nt 4 dU) | |

The sequences of CDNAs constructed to complementarily bind to the poly (A) tail region of eGFP mRNA are shown in Table 5 below.

TABLE 5

| SEQ ID | | |
|---|---|---|
| NO. | Name | Sequence |
| 24 | 3′ Poly (A) | TTTTTTTTTTTTTTTTTTTTTTTTTTTTTT |
| | tail cDNA | |
| | (30 nt) | |
| 25 | 5′ cap- | TTCTUACUCTTCUTTTCUCTCTUATTUCCCUCTUTTTUTTTTUTTTTUTTT |
| | poly(A) tail | T |
| | cDNA (52 nt | |
| | 11 dU) | |

The sequences of CDNAs constructed to complementarily bind to the 5′ cap region of eGFP mRNA and to have additional nucleotide modifications are shown in Table 6 below.

TABLE 6

| SEQ ID | | |
|---|---|---|
| NO. | Name | Sequence |
| 26 | 5′ cap cDNA | CTTACTCTTCTTTTCUCTCTTATTTCCCTC |
| | (Mod 0) | |
| 27 | 5′ cap cDNA | CTTACTCTTCTTTU_{OMe}C_{OMe}UC_{OMe}U_{OMe}CTTATTTCCCTC |
| | (Mod ±1) | |
| 28 | 5′ cap cDNA | CTTACTCTTCTTU_{OMe}U_{OMe}CUCU_{OMe}C_{OMe}TTATTTCCCTC |
| | (Mod ±2) | |
| 29 | 5′ cap cDNA | CTTACTCTTCTU_{OMe}U_{OMe}TCUCTC_{OMe}U_{OMe}TATTTCCCTC |
| | (Mod ±3) | |
| 30 | 5′ cap cDNA | CTTACTCTTCU_{OMe}U_{OMe}TTCUCTCU_{OMe}U_{OMe}ATTTCCCTC |
| | (Mod ±4) | |
| 31 | 5′ cap cDNA | CTTACTCTTCTU_{OMe}*U_{OMe}* TCUCT*C_{OMe}*U_{OMe}TATTTCCCTC |
| | (Mod ±3 +S*) | |

*phosphorothioate bond

### EXAMPLE 3

### Protein Expression Inhibition Effect Depending on Site of eGFP mRNA to Which cDNA Binds

CDNAs having the nucleotide sequences of SEQ ID NOs: 6, 17, 22 and 24 were bound to the 5′ cap region, start codon region, 3′ UTR region and poly (A) tail region of eGFP mRNA, respectively. To this end, an aqueous solution containing 300 ng (1 equivalent) of mRNA and 10 equivalents of cDNA (poly (A) tail cDNA was used in 40 equivalents considering the range) was mixed, and then subjected to an annealing process consisting of heating at 65°° C. for 3 minutes and then cooling at 0° C. for 15 minutes, thereby binding the two molecules. At this time, the longer the cooling time (up to 6 hours), the higher the efficiency. After the resulting solution was transferred into a 384-well plate (Greiner Bio-One), wheat-germ extract (Promega) (final concentration: 40% v/v) was additionally added thereto, and the in vitro translation efficiency was measured.

The fluorescence intensity of eGFP produced during eGFP mRNA translation was evaluated by measuring fluorescence once per minute for 3 hours at wavelengths of 485/510 nm (excitation/emission) at 25° C. using a microplate reader (Varioskan Flash).

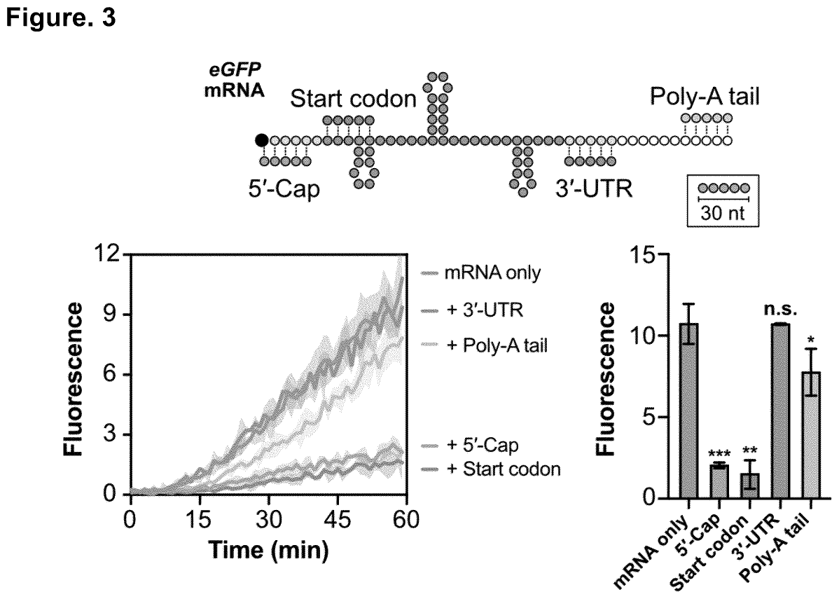

As a result, it was confirmed that the CDNAs complementary to the 5′ cap region and start codon region of eGFP mRNA, respectively, effectively restricted mRNA translation (FIG. 3).

### EXAMPLE 4

### Protein Expression Inhibitory Effect Depending on Nucleotide Length of 5′ Cap cDNA

The 5′ cap cDNA (10 nt) comprising the nucleotide sequence of SEQ ID NO: 11, the 5′ cap cDNA (20 nt) comprising the nucleotide sequence of SEQ ID NO: 12, the 5′ cap cDNA (30 nt) comprising the nucleotide sequence of SEQ ID NO: 6, and the 5′ cap cDNA (40 nt) comprising the nucleotide sequence of SEQ ID NO: 13, which were different in length, were each bound to the 5′ cap region of eGFP mRNA. To this end, an aqueous solution containing 300 ng (1 equivalent) of mRNA and 10 equivalents of DNA was mixed, and then subjected to an annealing process consisting of heating at 65° C. for 3 minutes and then cooling at 0°° C. for 15 minutes, thereby binding the two molecules. Thereafter, mRNA translation efficiency was measured in the same manner as in Example 3.

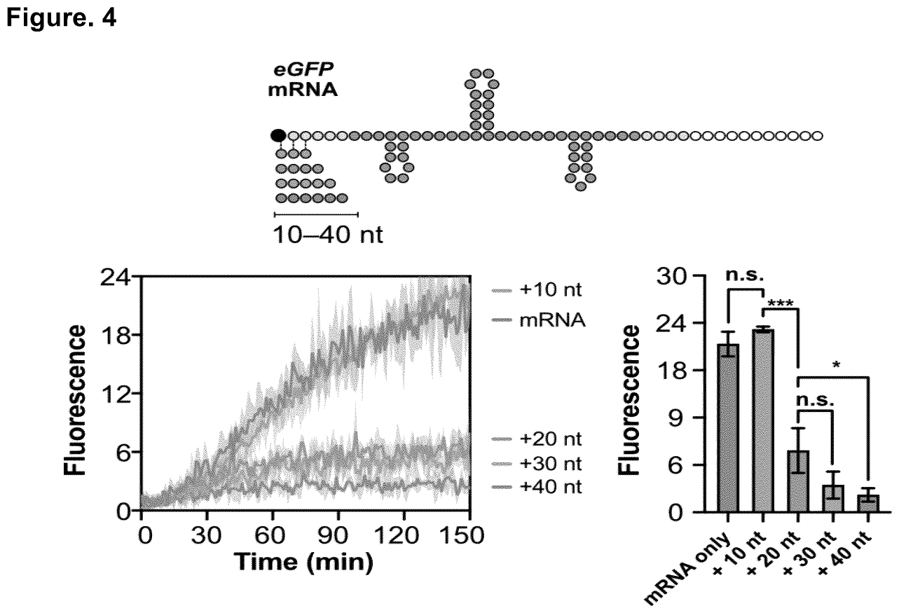

As a result, it was confirmed that the 5′ cap cDNA with a length of 20 nt or longer effectively inhibited mRNA translation (FIG. 4).

### EXAMPLE 5

### Protein Expression Restoration Effect of Use of CDNA Containing Modified Nucleotides and UDG, a Protein That Repairs the Same

When uridine DNA glycosylase (UDG), a protein that repairs dU, is used, the uracil base is removed by the repair mechanism, which weakens the binding force between CDNA and mRNA, allowing the mRNA to be released from the cDNA containing the modified base.

300 ng (1 equivalent) of eGFP mRNA was mixed with 10 equivalents of the 5′ cap cDNA (30 nt 7 dU) comprising the nucleotide sequence of SEQ ID NO: 10. The two molecules were bound together through an annealing process consisting of heating at 65° C. for 3 minutes and then cooling at 0°° C. for 15 minutes. The resulting solution was transferred into a 384-well plate, and each well was treated with 0 to 2.5 U of UDG (New England BioLabs, NEB), a DNA repair protein. Thereafter, in the same manner as in Example 3, wheat-germ extract (40% v/v) was additionally added, and the mRNA translation efficiency was measured.

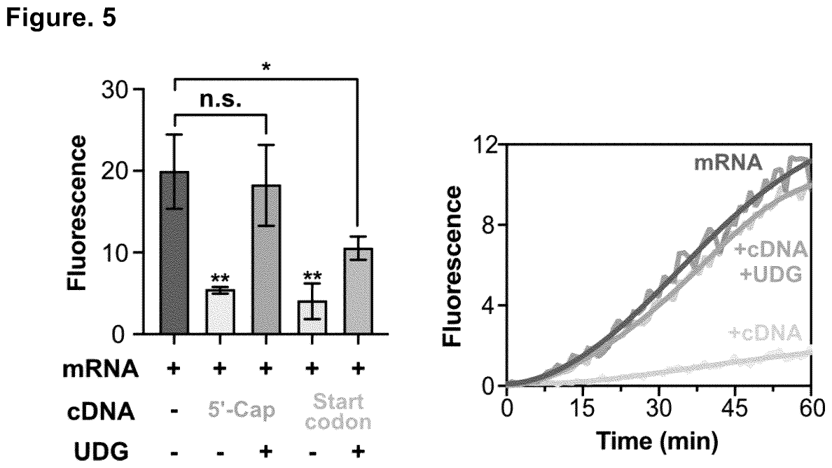

As a result, it was confirmed that the cDNA containing dU exhibited a protein expression inhibitory effect similar to that of cDNA containing dT, and in the presence of UDG, which repairs dU, DNA was repaired and mRNA was re-released by 95% or more (FIG. 5).

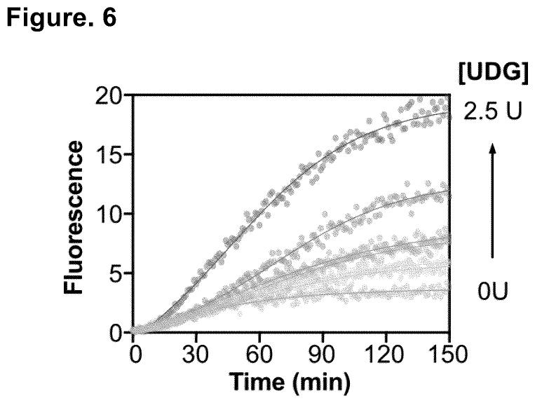

In addition, it was confirmed that, when the concentration of UDG was controlled within 0 U to 2.5 U, the time for DNA repair could be controlled depending on the repair activity of UDG, which is directly related to the mRNA release rate, suggesting that the protein expression rate can be controlled (FIG. 6).

Additionally, the 5′ cap cDNA (30 nt 3 dU) comprising the nucleotide sequence of SEQ ID NO: 7, the 5′ cap cDNA (30 nt 4 dU) comprising the nucleotide sequence of SEQ ID NO: 8,the 5′ cap cDNA (30 nt 5 dU) comprising the nucleotide sequence of SEQ ID NO: 9, and the 5′ cap cDNA (30 nt 7 dU) comprising the nucleotide sequence of SEQ ID NO: 10 were each bound to eGFP mRNA. To this end, an aqueous solution containing 300 ng (1 equivalent) of mRNA and 5 equivalents of CDNA was mixed, and then subjected to an annealing process consisting of heating at 65° C. for 3 minutes and cooling at 0°° C. for 15 minutes, thereby binding the two molecules. After the resulting solution was transferred into a 384-well plate, 2.5 U of UDG and wheat-germ extract (40% v/v) was additionally added thereto, and then the mRNA translation efficiency was measured in the same manner as in Example 3.

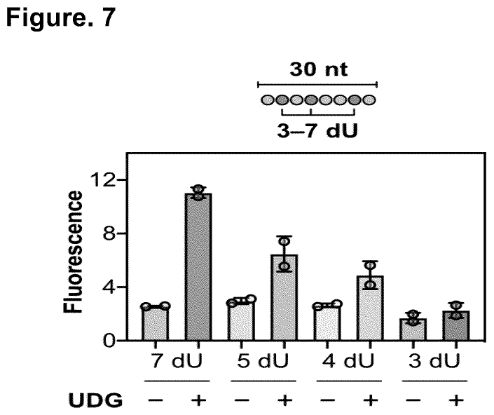

As a result, it was confirmed that cDNA with a larger number of modified nucleotides, i.e., cDNA with a larger number of dU nucleotides, exhibited a higher protein expression rate (FIG. 7).

### EXAMPLE 6

### Protein Expression Inhibitory Effect Depending on Nucleotide Length of 5′ Cap cDNA Containing Modified Nucleotides

For in vivo experiments, 20% Hela cells were cultured with 100 μL of growth media (DMEM containing 10% FBS, 1% pen/strep; Gibco) in each well of a 96-well plate at 37° C. under 5% CO₂. After one day, when the cell confluency reached approximately 40%, the 5′ cap cDNA (30 nt 7 dU) comprising the nucleotide sequence of SEQ ID NO: 10 and the 5′ cap cDNA (60 nt 13 dU) comprising the nucleotide sequence of SEQ ID NO: 16 were each bound to 40 ng of eGFP mRNA. To this end, an aqueous solution containing 300 ng (1 equivalent) of mRNA and 5 equivalents of DNA was mixed, and then subjected to an annealing process consisting of heating at 65°° C. for 3 minutes and then cooling at 0°° C. for 15 minutes, thereby binding the two molecules. The resulting aqueous solution was mixed with Lipofectamine MessengerMAX (Thermo Fisher) at room temperature for 5 minutes, and then transfected into 40% HeLa cells in each well. At 6, 12 and 24 hours after transfection, in vivo translation was evaluated by measuring fluorescence at wavelengths of 485/510 nm at 37° C. using a microplate reader device.

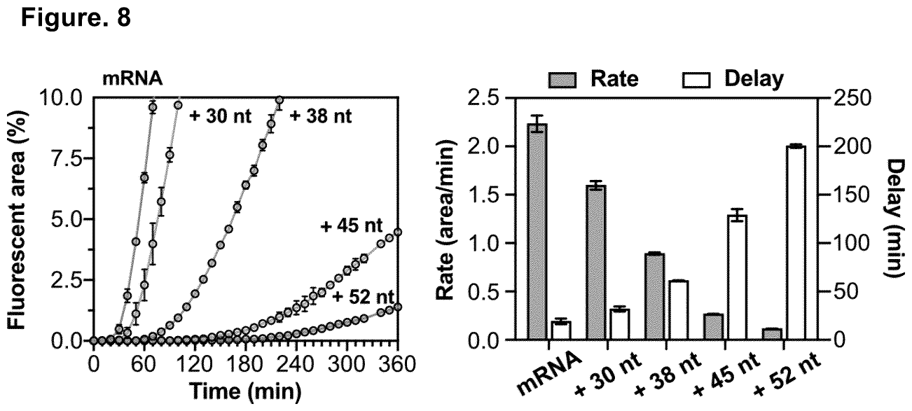

As a result, it was confirmed that the protein expression rate could be controlled by controlling the nucleotide length of CDNA (FIG. 8).

Additionally, 20% Hela cells were cultured with 110 μL growth media (DMEM containing 10% FBS, 1% pen/strep; Gibco) in FD35 fluorodish (World Precision Instruments) at 37° C. under 5% CO₂. After one day, when the cell confluency reached approximately 40%, the 5′ cap cDNA (30 nt 7 dU) comprising the nucleotide sequence of SEQ ID NO: 10 and the 5′ cap cDNA (60 nt 13 dU) comprising the nucleotide sequence of SEQ ID NO: 16 were each bound to 40 ng of eGFP mRNA. To this end, an aqueous solution containing 300 ng (1 equivalent) of mRNA and 5 equivalents of CDNA was mixed, and then subjected to an annealing process consisting of heating at 65° C. for 3 minutes and then cooling at 0° C. for 15 minutes, thereby binding the two molecules. The resulting aqueous solution was mixed with Lipofectamine MessengerMAX (Thermo Fisher) at room temperature for 5 minutes, and then transfected into 40% HeLa cells in each well. At 6 and 24 hours after transfection, the growth media was removed, and the cells were washed three times with PBS (Gibco). Thereafter, to stain the cell nucleus, the cells were treated with 3 μg/mL Hoechst 33342 (Sigma Aldrich) in 110 μL of growth media and then incubated for 30 minutes at 37° C. under 5% CO₂. Next, the cells were washed again with PBS, and then fixed with 110 μL of 4% formaldehyde in PBS for 30 minutes at room temperature. Then, the PBS was replaced with fresh PBS. DAPI and eGFP images were obtained using confocal microscopy (Nikon).

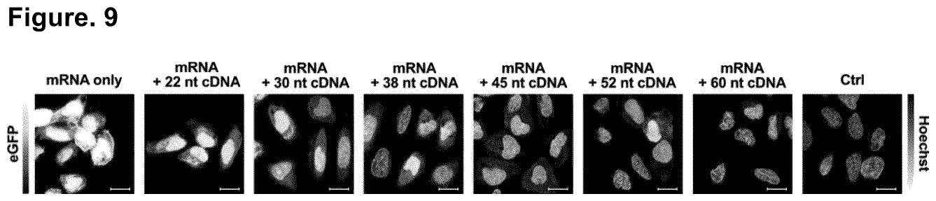

As a result of fluorescence microscopy observations 6 and 12 hours after transfection, it was confirmed that the protein expression rate could be controlled by controlling the nucleotide length of CDNA (FIG. 9).

### EXAMPLE 7

### Protein Expression Restoration Effect of Use of 5′ Cap cDNA Containing Modified Nucleotides and UDG, a Protein that Repairs the Same

When the 5′ cap region of mRNA is bound with cDNA to inhibit protein expression and then the modified nucleotides in the cDNA are removed using a DNA repair enzyme, the mRNA is separated from the cDNA and protein expression is restored.

Additionally, when the surrounding nucleotides of the CDNA used are modified, the cDNA may have the effect of increasing the in vivo stability of mRNA by inhibiting exonuclease-induced degradation of the mRNA during delayed expression.

CDNAs of SEQ ID NOS: 26 to 30, which have different distances between dUs and in which the sugar is modified with a 2′-methoxy group, and cDNA of SEQ ID NO: 31, in which both the sugar and phosphate are modified, were each bound to the 5′ cap region of eGFP mRNA.

To this end, an aqueous solution containing 300 ng (1 equivalent) of mRNA and 5 equivalents of cDNA was mixed, and then subjected to an annealing process consisting of heating at 65° C. for 3 minutes and then cooling at 0°° C. for 15 minutes, thereby binding the two molecules. Thereafter, mRNA translation efficiency was measured in the same manner as in Example 3.

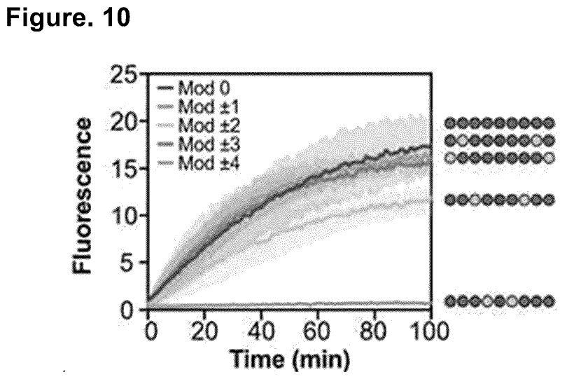

As a result, it was confirmed that, when at least two unmodified DNAs were included around dU, they did not affect the repair mechanism, and in the presence of UDG, which repairs dU, DNA was repaired and mRNA was re-released (FIG. 10).

### EXAMPLE 8

### Enzymatic Degradation Inhibitory Effect of 5′ Cap-Poly (A) Tail cDNA

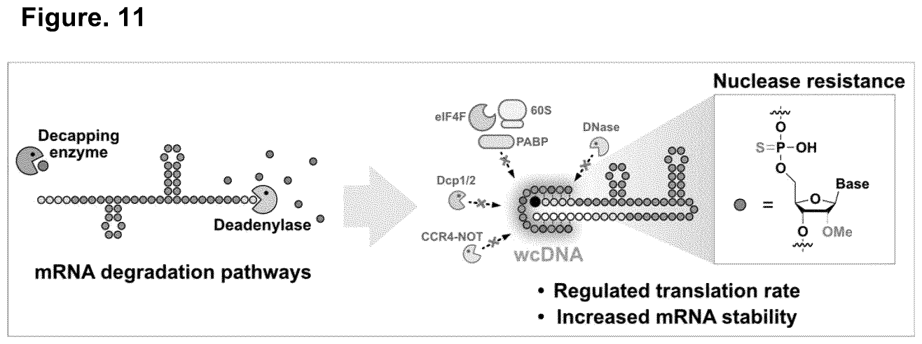

When a cDNA structure is constructed so that it may bind to the 5′ cap region and the poly (A) tail region of mRNA, it may enhance the stability of the mRNA by inhibiting the interaction between decapping enzymes that remove the 5′ cap and exonuclease that degrades the poly (A) tail (FIG. 11).

The 5′ cap-poly (A) tail cDNA (52 nt 11 dU) comprising the nucleotide sequence of SEQ ID NO: 25 was bound to eGFP mRNA. To this end, NEBuffer 4 solution containing 300 ng (1 equivalent) of mRNA and 5 equivalents of cDNA was mixed, and then subjected to an annealing process consisting of heating at 65° C. for 3 minutes and then cooling at 0° C. for 5 hours, thereby binding the two molecules. 1 U of exonuclease T (NEB), a type of 3′ exonuclease that degrades the poly (A) tail, was added thereto, followed by incubation at 25° C. for 10, 20, and 30 minutes. Immediately after incubation, 6X DNA loading dye (Thermo Fisher) diluted to 1X was added thereto, and the resulting mixture was loaded onto a 1.5% agarose gel stained with SYBR safe (Thermo Fisher). After gel electrophoresis at 100 V for 50 min, a gel image was obtained using Chemidoc (Bio-Rad Laboratories).

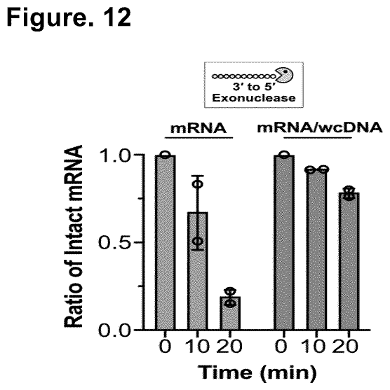

As a result, it is suggested that, when cDNA is constructed so that it may bind to the 5′ cap and poly (A) tail regions, it may enhance the stability of the mRNA (FIG. 12).

What is claimed is:

1. An mRNA composition comprising:
an mRNA sequence comprising, in order from 5′ to 3′, a 5′ cap region, a 5′ UTR region, a start codon region, a 3′ UTR region, and a poly (A) tail region; and
an antisense oligonucleotide containing a region complementary to the 5′ cap region.

2. The MRNA composition according to claim 1, wherein the mRNA is complexed with one or more lipids to form lipid nanoparticles or liposomes.

3. The mRNA composition according to claim 2, wherein the lipid nanoparticles comprise a cationic lipid, a PEG-modified lipid, a sterol, and a non-cationic lipid.

4. The mRNA composition according to claim 1, wherein the antisense oligonucleotide is one in which 1 to 15 thymines are substituted with uracil.

5. The mRNA composition according to claim 1, wherein the antisense oligonucleotide comprises a nucleotide sequence selected from the group consisting of SEQ ID NOS: 6 to 16.

6. The mRNA composition according to claim 1, wherein the antisense oligonucleotide is one in which 1 to 5 nucleotides are methoxylated.

7. mRNA composition according to claim 6, wherein the antisense oligonucleotide comprises a nucleotide sequence selected from the group consisting of SEQ ID NOS: 26 to 31.

8. The mRNA composition according to claim 1, further comprising an antisense oligonucleotide comprising a nucleotide sequence selected from the group consisting of SEQ ID NOs: 17 to 21.

9. The mRNA composition according to claim 1, further comprising an antisense oligonucleotide comprising the nucleotide sequence of SEQ ID NO: 22 or 23.

10. The mRNA composition according to claim 1, further comprising an antisense oligonucleotide comprising the nucleotide sequence SEQ ID NO: 24 or 25.

11. The mRNA composition according to claim 1, wherein the antisense oligonucleotide comprises a fragment of 10 to 60 nucleotides (nt).

12. The mRNA composition according to claim 1, further comprising at least one adjuvant or active agent.
